(19) United States
(12) Patent Application Publication
(10) Pub. No.: US 2021/0304900 A1
(43) Pub. Date: Sep. 30, 2021

(54) METHOD AND APPARATUS FOR PREDICTING TRANSMISSION OF AN INFECTIOUS DISEASE, COMPUTER APPARATUS AND STORAGE MEDIUM

(71) Applicant: SOUTH UNIVERSITY OF SCIENCE AND TECHNOLOGY OF CHINA, Shenzhen (CN)

(72) Inventors: Xuan Song, Shenzhen (CN); Zipei Fan, Shenzhen (CN); Zhiwen Zhang, Shenzhen (CN); Chuang Yang, Shenzhen (CN); Yinghao Liu, Shenzhen (CN); Renhe Jiang, Shenzhen (CN); Quanjun Chen, Shenzhen (CN); Ryosuke Shibasaki, Shenzhen (CN)

(21) Appl. No.: 16/928,689

(22) Filed: Jul. 14, 2020

(30) Foreign Application Priority Data
Mar. 31, 2020 (CN) 202010242822.6

(51) Int. Cl. G16H 50/80; G16H 50/70

(52) U.S. Cl. CPC G16H 50/80; G16H 50/70

(57) ABSTRACT

A method and apparatus for predicting transmission of an infectious disease, a computer apparatus, and a storage medium. The method includes determining one or more objects to be predicted, matching in a historical travel path database for a corresponding predicted path of each of the one or more objects to be predicted, and determining a transmission trend of the infectious disease in the one or more objects to be predicted based on the predicted travel paths. The predicted travel path of each object to be predicted is matched in the historical travel path database thus achieving the effect of reducing the computation amount of obtaining the predicted travel path.

### CROSS-REFERENCE TO RELATED APPLICATION

This patent application claims priority to China patent application No. 202010242822.6 filed on Mar. 31, 2020, the disclosure of which is hereby incorporated herein by reference in its entirety.

### TECHNICAL FIELD

Embodiments of the present disclosure relate to the technical field of infectious disease prevention, and more particularly relate to a method and apparatus for predicting transmission of an infectious disease, a computer apparatus, and a storage medium.

### BACKGROUND

The simulation and prediction of people flow has important significance and plays an important role in urban planning and response to major emergency events.

In current method for prediction of transmission of an infectious disease, a predicted travel path of each person in the people flow is obtained, including the predicted travel path of an infected person and the predicted travel path of an uninfected person. Then, the transmission trend of the infectious disease is predicted based on the predicted travel paths of all the people. Common methods for predicting a travel path typically consist in performing computation using physics-based dynamical models, classical multi-agent model in civil engineering and computer science, generative models in the field of machine learning, or the like.

However, these models require individually computing each user's travel path and modelling the influence between the users, thus posing extremely high requirements on the amount of computation.

### SUMMARY

Embodiments of the present disclosure provide a method and apparatus for predicting transmission of an infectious disease, a computer apparatus and a storage medium to reduce the computation for acquiring a predicted travel path. In a first aspect, an embodiment of the present disclosure provides a method for predicting transmission of an infectious disease. The method includes the following operations:

determining one or more objects to be predicted;

matching in a historical travel path database for a corresponding predicted travel path of each of the one or more objects to be predicted; and

determining a transmission trend of the infectious disease in the one or more objects to be predicted based on the predicted travel paths.

Optionally, the historical travel path database includes respective historical travel paths of different users in each historical time period within a time cycle, and the operation of matching in the historical travel path database for the corresponding predicted path of each of the one or more objects to be predicted includes the following operations:

obtaining a current time period and determining a next time period corresponding to the current time period;

determining in the time cycle a target historical time period matching the next time period;

matching in the historical travel path database for a corresponding historical travel path of each of the one or more objects to be predicted in the target historical time period; and using the corresponding historical travel path of each of the one or more objects to be predicted in the target historical time period as the corresponding predicted travel path of each of the one or more objects to be predicted.

Optionally, the historical travel path database further includes respective identifiers of the different users, and the operation of matching in the historical travel path database the corresponding predicted path of each object to be predicted includes the following operations:

obtaining a corresponding target identifier of each of the one or more objects to be predicted;

determining whether the historical travel path database comprises a matching identifier that matches the target identifier;

in response to determining that the historical travel path database comprises the matching identifier that matches the target identifier, using the historical travel path of a corresponding user of the matching identifier in the target historical time period as the corresponding predicted travel path of each of the one or more objects to be predicted in the target historical time period; and

in response to determining that the historical travel path database does not comprise a matching identifier that matches the target identifier, determining a similarity between each of the different users and the object to be predicted; and

selecting the corresponding historical travel path of the user having the greatest similarity in the target historical time period, as the corresponding historical travel path of each of the one or more objects to be predicted in the target historical time period.

Optionally, before the operation of matching in the historical travel path database for the corresponding predicted path of each object to be predicted, the method includes the following operations:

obtaining location data of the different users;

using the respective location data of each of the different users as the corresponding historical travel path of each of the different users; and

for each of the historical time periods within the time cycle, storing the corresponding historical travel path of each of the different users and the corresponding identifier of each of the different users in association to obtain the historical travel path database.

Optionally, the operation of determining the transmission trend of the infectious disease in the one or more objects to be predicted based on the predicted travel paths includes the following operations:

obtaining a pre-made control plan;

determining affected objects and unaffected objects among the one or more objects to be predicted based on the control plan;

matching in the historical travel path database for respective simulated travel paths of the affected objects based on respective predicted travel paths of the affected objects; and

determining the transmission trend of the infectious disease in the affected objects and the unaffected objects based on the respective simulated travel paths of the affected object and the respective predicted travel paths of the unaffected objects.

Optionally, the operation of determining the transmission trend of the infectious disease in the one or more objects to be predicted based on the predicted travel paths includes the following operations:

determining an infected object, a virus carrier, a recovered patient, and an uninfected object among the one or more objects to be predicted; and

performing computation on a corresponding predicted path of the infected object, a corresponding predicted path of the virus carrier, a corresponding predicted path of the recovered patient, and a corresponding predicted path of the uninfected object using an SEIR (Susceptible, Exposed, Infectious, Recovered) infection model to obtain the transmission trend of the infectious disease in the uninfected objects.

Optionally, the method further includes the following operation:

displaying the transmission trend in a visual form.

In a second aspect, an embodiment of the present disclosure provides an apparatus for predicting transmission of an infectious disease. The apparatus includes an object-to-be-predicted determination module, a predicted-travel-path matching module, and a transmission-trend determination module.

The object-to-be-predicted determination module is configured to determine one or more objects to be predicted.

The predicted-travel-path matching module is configured to match in a historical travel path database for a corresponding predicted path of each of the one or more objects to be predicted.

The transmission-trend determination module is configured to determine a transmission trend of the infectious disease in the one or more objects to be predicted based on the predicted travel path.

In a third aspect, an embodiment of the present disclosure further provides a computer apparatus.

The computer apparatus includes one or more processors.

The computer apparatus further includes a storage device. The storage device is configured to store one or more computer programs.

When the one or more computer programs are executed by the one or more processors, the one or more processors are caused to perform the method for predicting transmission of an infectious disease of any embodiment of the present disclosure.

In a fourth aspect, an embodiment of the present disclosure provides a computer-readable storage medium storing a computer program. When the computer program is executed by a processor, the method for predicting transmission of an infectious disease of any embodiment of the present disclosure is performed.

According to the embodiments of the present disclosure, one or more objects to be predicted are determined, a corresponding predicted path of each object to be predicted is matched for in a historical travel path database, and a transmission trend of the infectious disease in the one or more objects to be predicted is determined based on the predicted travel paths. This solves the problem that the existing models require individually computing each user's travel path and modelling the influence between the users, which pose extremely high requirements on the amount of computation, thereby achieving the effect of reducing the computation amount for obtaining the predicted travel path.

### BRIEF DESCRIPTION OF DRAWINGS

FIG. 1 is a flowchart of a method for predicting transmission of an infectious disease according to Embodiment one of the present disclosure.

FIG. 2 is a flowchart of a method for predicting transmission of an infectious disease according to Embodiment two of the present disclosure.

FIG. 3 is a block diagram of an apparatus for predicting transmission of an infectious disease according to Embodiment three of the present disclosure.

FIG. 4 is a schematic diagram of a computer apparatus according to Embodiment five of the present disclosure.

### DETAILED DESCRIPTION

Hereinafter the present disclosure will be described in further detail in conjunction with the drawings and embodiments. It is to be understood that the specific embodiments set forth below are intended to illustrate rather than limiting the present disclosure. Additionally, for ease of description, only part, not all, of the structures related to the present disclosure are illustrated in the drawings.

Before the exemplary embodiments are discussed in more detail, it is to be noted that part of the exemplary embodiments are described as processing or methods depicted in flowcharts. Although the flowcharts describe the steps as sequentially processed, many of the steps may be implemented concurrently, coincidently, or simultaneously. Additionally, the sequence of the steps may be rearranged. The processing may be terminated when operations of the processing are completed, but may further have additional steps not included in the drawings. The processing may correspond to a method, a function, a procedure, a subroutine, a subprogram or the like.

Furthermore, the terms “first”, “second” or the like may be used herein to describe various directions, acts, steps, elements or the like, but these directions, acts, steps or elements are not limited by these terms. These terms are merely used to distinguish one direction, action, step or element from another direction, action, step or element. For example, without departing from the scope of the present application, first information may be referred to as second information, and similarly, the second information may be referred to as the first information. The first information and the second information are both information, but not the same information. Terms such as “first”, “second” are not to be construed as indicating or implying relative importance or implicitly indicating the number of technical features as indicated. Thus, a feature defined as a “first” feature or a “second” feature may explicitly or implicitly include one or more of such features. As used herein, the term “multiple” is defined as at least two, for example, two or three, unless otherwise specified and limited.

### Embodiment One

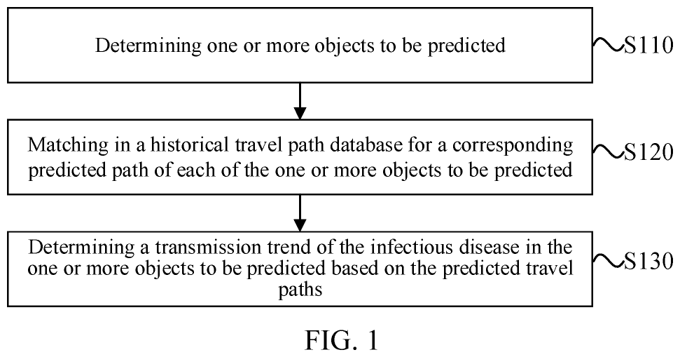

FIG. 1 is a flowchart of a method for predicting transmission of an infectious disease according to embodiment one of the present disclosure. The method is available to a scenario of predicting a transmission trend of the infectious disease. The method may be performed by an apparatus for predicting transmission of an infectious disease. The apparatus may be implemented in software and/or hardware and may be integrated in a computer apparatus.

As illustrated in FIG. 1, the method for predicting transmission of an infectious disease according to Embodiment one of the present disclosure includes the steps described below.

In S110, one or more objects to be predicted are determined.

An object to be predicted refers to an individual, an animal or the like involved in the prediction of infectious disease transmission, for example, people involved in the infectious disease transmission. Optionally, all people in a preset region may be selected as objects to be predicted in this embodiment. The preset region may be selected artificially. Optionally, preset regions may be divided according to administrative regions, geographical natures or the like, which is not specifically limited here. For example, all people in China are selected as the objects to be predicted in this embodiment, or all people in Hubei are selected as the objects to be predicted in this embodiment, which may be selected according to needs and is not specifically limited herein.

In this embodiment, the object to be predicted may include all people without a control plan, or may include an affected object and an unaffected object under the control plan. The control plan may be added according to needs.

In S120, a corresponding predicted path of each object to be predicted is matched for in a historical travel path database.

The historical travel path database stores historical travel paths of different users. A historical travel path refers to a travel route in the past and already exists. The predicted travel path refers to a travel route of the object to be predicted in the future. The predicted travel path has not occurred and is regarded as the travel path most likely to occur for the object to be predicted. Specifically, each object to be predicted individually corresponds to one or more predicted travel paths. The predicted travel path of the object to be predicted is matched in the historical travel path database so that a historical travel path in the historical travel path database is used as the predicted travel path of the object to be predicted. This greatly reduces computation.

In an optional embodiment, the historical travel path database includes historical travel paths of the different users and corresponding to historical time periods within a time cycle, and the step of matching the corresponding predicted path of each object to be predicted in the historical travel path database includes the steps described below.

A current time period is acquired, and a next time period corresponding to the current time period is determined. A target historical time period matching the next time period is determined in the time cycle. A historical travel path corresponding to each object to be predicted in the target historical time period is matched in the historical travel path database. The historical travel path corresponding to each object to be predicted in the target historical time period is determined as the corresponding predicted path of each object to be predicted.

In this embodiment, the time cycle refers to a cycle time interval. For example, the time cycle may be from the 1^{st} to the 31^{st} in a month, or may be from Monday to Sunday, or may be a day, which is not specifically limited. In response to a time cycle from the 1^{st} to the 31^{st} in a month, each historical time period within the time cycle may be a day, for example, the 1^{st} in the month is one historical time period and the 2^{nd} in the month is another historical time period; in response to a time cycle from Monday to Sunday, Monday may be one historical time period and Tuesday may be another historical time period; in response to a time cycle of a day, the historical time period may each hour, for example, 8 o'clock is one historical time period and 9 o'clock is another historical time period; which is not specific limited here and may be configured according to needs. In this embodiment, each historical time period within the time cycle corresponds to a respective historical travel path.

In this embodiment, the current time period refers to a time at present. The current time period is determined according to the form of the time cycle. For example, in response to the time cycle from the 1^{st} to the 31^{st} in a month, the current time period may be each day, for example, the current time period is the 1^{st} in the month; in response to the time cycle from Monday to Sunday, the current time period may be Monday; which is not limited here. The specific current time period is determined according to actual conditions. The next time period refers to the next time period of the current time period. The current time period is determined according to the form of the time cycle. For example, in response to the time cycle from the 1^{st} to the 31^{st} in a month, if the current time period is March 1, the next time period is March 2, which is not specifically limited here. The target historical time period refers to a historical time period that matches the next time period in the time cycle. For example, in response to the next time period of March 2, the target historical time period is the 2^{nd} in a month. In this embodiment, the historical travel path corresponding to the target time of the object to be predicted is used as the predicted travel path of the object to be predicted.

In an optional embodiment, the historical travel path database further includes respective identifiers of the different users, and the step of matching the corresponding predicted path of each object to be predicted in the historical travel path database includes the steps described below.

A target identifier corresponding to each object to be predicted is acquired. It is determined whether the historical travel path database has a matching identifier matching the target identifier. In response to the historical travel path database having the matching identifier matching the target identifier, a historical travel path of a user corresponding to the matching identifier in the target historical time period is determined as the corresponding predicted path of each object to be predicted in the target historical time period. In response to the historical travel path database having no matching identifier matching the target identifier, similarity between each of the different users and each object to be predicted is determined, and a historical travel path corresponding to a user having the greatest similarity in the target historical time period is selected and determined as the historical travel path corresponding to each object to be predicted in the target historical time period.

An identifier refers to information that can reflect the unique identity of a user. For example, the identifier may take the form of name+identity card, or each user is allocated a unique serial number, which is not specifically limited here. The target identifier refers to an identifier of the object to be predicted.

Specifically, in response to the historical travel path database having the matching identifier matching a target user, it is indicated that the historical travel path database includes a historical travel path of the object to be predicted indicated by the target identifier, and a historical travel path corresponding to the matching identifier may be used as the historical travel path of the object to be predicted. For example, if the object to be predicted has a target identifier of A and the history database also includes a matching identifier A, then a history travel path corresponding to the matching identifier A is used as the history travel path of the object to be predicted. In response to the historical travel path database having no matching identifier matching the target identifier, the similarity between each of the different users and the object to be predicted is determined. Specifically, in response to the historical travel path database having no matching identifier matching the target identifier, it is indicated that the historical travel path database does not have the historical travel path of the object to be predicted indicated by the target identifier. Optionally, information about the object to be predicted may be acquired and compared with information about each user in the historical travel path database, and the similarity between the information about the object to be predicted and the information about each user is used as the similarity between each user and the object to be predicted in this embodiment. Optionally, a historical travel path of the object to be predicted at the last time may be acquired and compared with a historical travel path of each user at the last time in the historical travel path database, and the coincidence degree between the travel paths is used as the similarity between each user and the object to be predicted in this embodiment. The historical travel path corresponding to the user having the greatest similarity in the target historical time period is selected and determined as the historical travel path corresponding to the object to be predicted in the target historical time period.

In an optional embodiment, before the step of matching the corresponding predicted path of each object to be predicted in the historical travel path database, the method includes the steps described below.

Location data of the different users is acquired. Location data corresponding to each user is determined as a historical travel path corresponding to each user. According to each historical time period within the time cycle, the historical travel path corresponding to each user and an identifier corresponding to each user are stored in association to obtain the historical travel path database.

The location data refers to data of a user at different locations. Optionally, locating information about a mobile terminal used by the user may be used as the location data in this embodiment. Specifically, in response to the location data being a complete travel path, the location data is directly used as the historical travel path corresponding to each user; in response to the location data being some discrete location points and including some null values or abnormal values, the null values and the abnormal values are removed through data cleaning, and a complete travel path is fitted with the discrete location points and used as the historical travel path corresponding to each user. Moreover, the historical travel path and the identifier corresponding to each user are stored in association according to each historical time period within the time cycle.

In step S130, a transmission trend of the infectious disease in the one or more objects to be predicted is determined according to the predicted travel path.

The transmission trend refers to a trend of the infectious diseases to spread and infect among the objects to be predicted. Optionally, the transmission trend may be displayed in a visual form so that the predicted result of the transmission trend is visually reflected. Optionally, a transmission trend within a set region may be predicted. For example, it is feasible to predict a transmission trend in Wuhan, a transmission trend in the entire China, or the like, which is not specifically limited here.

In an optional embodiment, the step of determining the transmission trend of the infectious disease in the one or more objects to be predicted according to the predicted travel path includes the steps described below.

An infected object, a virus carrier, a recovered patient and an uninfected object among the one or more objects to be predicted are determined. Computation is performed on a corresponding predicted path of the infected object, a corresponding predicted path of the virus carrier, a corresponding predicted path of the recovered patient, and a corresponding predicted path of the uninfected object through an SEIR infection model to obtain a transmission trend of the infectious disease in uninfected objects.

The infected object is an object regarded to be infected with the infectious disease and have symptoms. The virus carrier is an object carrying a virus but in a latent period. The uninfected object is an object regarded to be not infected with the infectious disease. The recovered patient is an object self-healed or treated to acquire immunity. Specifically, existing objects confirmed to be infected with the infectious disease and already having symptoms are regarded as infected objects, objects detected and confirmed to carry the virus and have no symptoms in the latent period are regarded as virus carriers, objects self-healed or treated to acquire immunity are regarded as recovered patients, and others are uninfected objects. These may be determined according to medical diagnosis data, or the distribution of existing confirmed cases, where a part of the objects to be predicted corresponding to the distribution may be selected from among the objects to be predicted to serve as the infected objects, the virus carriers, the recovered patients and the like, and other objects may be uninfected objects, which may be selected according to needs and not specifically limited here. The SEIR model is a transmission model and abstractly describes an information transmission process. The SEIR model is the most classical model in infectious disease models. The corresponding predicted path of the infected object and the corresponding predicted path of the uninfected object are used as input parameters and inputted to the SEIR infection model to perform computation, so that the transmission trend of the infectious disease in the uninfected object is obtained. Optionally, the SIR model may be selected for simulation according to needs, which is not limited here. The virus carrier is not considered in the SIR model, and an appropriate model may be selected for prediction according to specific features of the infectious diseases, which is not limited here. Specifically, the SEIR model may be used for training to obtain a pre-trained trend model, so that a specific transmission trend is predicted.

According to the technical solution of this embodiment of the present disclosure, one or more objects to be predicted are determined, a corresponding predicted path of each object to be predicted is matched in a historical travel path database, and a transmission trend of the infectious disease in the one or more objects to be predicted is determined according to the predicted travel path. Since the predicted travel path is obtained through the matching in the historical travel path database, it not necessary to perform computation by using various models, thus greatly reducing the computation and achieving the technical effect of reducing the computation for acquiring the predicted travel path.

### Embodiment Two

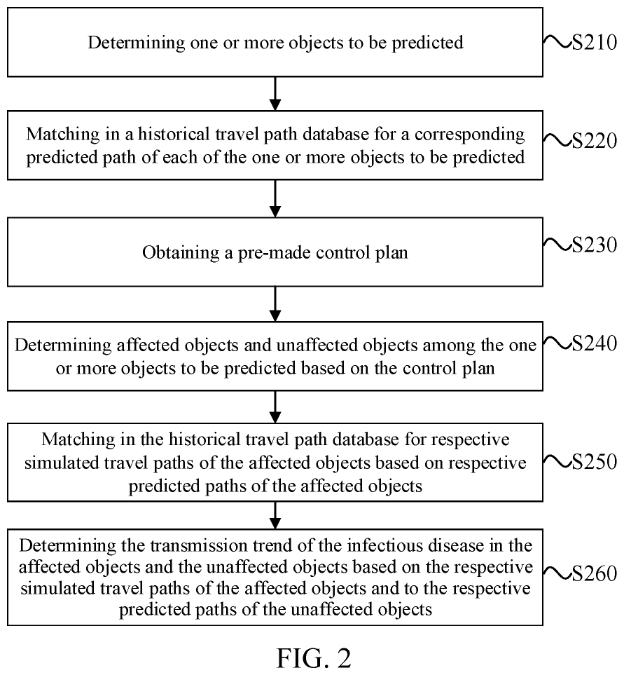

FIG. 2 is a flowchart of a method for predicting transmission of an infectious disease according to Embodiment two of the present disclosure. This embodiment is a further refinement of the preceding technical solution and is suitable for a scenario of predicting a transmission trend of the infectious diseases under different control plans. The method may be executed by an apparatus for predicting transmission of an infectious disease. The apparatus is implemented in software and/or hardware and may be integrated in a server.

As illustrated in FIG. 2, the method for predicting transmission of an infectious disease according to Embodiment two of the present disclosure includes the steps described below.

In S210, one or more objects to be predicted are determined.

An object to be predicted refers to an individual, an animal or the like involved in the prediction of the infectious disease transmission, for example, people involved in the infectious disease transmission. Optionally, all people in a preset region may be selected as objects to be predicted in this embodiment. The preset region may be selected artificially.

In S220, a corresponding predicted path of each object to be predicted is matched for in a historical travel path database.

The historical travel path database stores historical travel paths of different users. A historical travel path refers to a travel route in the past time and already exists. The predicted travel path refers to a travel route of the object to be predicted in the future. The predicted travel path has not occurred and is regarded as the travel path most likely to occur for the object to be predicted. Specifically, each object to be predicted individually corresponds to one or more predicted travel paths.

In step S230, a pre-made control plan is obtained.

The control plan refers to a plan for controlling the object to be predicted, and is configured to control the travel of the object to be predicted. Optionally, the control plan includes traffic control, city blockade, regional evacuation and other strategies, which are not specifically limited here. For example, the control plan may be increasing/decreasing by a certain percentage on the basis of roads/administrative districts/specific functional region types, such as stations and large commercial blockade regions, or on the basis of traffic flow in a certain region.

In step S240, an affected object and an unaffected object among the one or more objects to be predicted are determined based on the control plan.

The affected object refers to people affected by the control plan among the objects to be predicted. For example, if an object to be predicted has a predicted travel path of leaving the city while the control plan is blockading the city, then the object to be predicted is the affected object in this embodiment. Optionally, an object to be predicted corresponding to a travel type conflicting with the control plan may be used as the affected object according to a travel type associated with the predicted travel path of the object to be predicted. The travel type associated with the predicted travel path may be determined by a road network matched with the predicted travel path. The road network refers to a road system composed of various roads and interwoven into a network distribution in a certain region. For example, if the object to be predicted corresponds to a predicted travel path of being on an expressway between cities, then the predicted travel path of the object to be predicted is associated with a travel type of leaving the city.

Affected objects are people whose behaviors are changed under a control strategy. For example, the city blockade control affects all people who need to leave and enter the city, and the closure of entertainment venues affects all people who will enter entertainment venues. Main reasons for the reduction in computation here are: 1) we only focus on and simulate people affected by the control strategy instead of all people, and 2) we extract travel paths from the historical travel path database, thus avoiding a large amount of computation to generate a brand-new travel path.

The infectious disease is simulated among all people including unaffected people and affected people. We firstly use the data of a similar day from historical travel paths as a basic simulated result (the basic simulated result assumes that there is no great change in people flow, for example, in the simulation of a travel path of Tuesday, we firstly find a travel path of Tuesday in the historical data to serve as the basic simulated result). According to the control strategy, we may distinguish the people unaffected by the control from the people affected by the control. For the people unaffected by control, we do not modify travel paths of the people. For the people affected by the control, a conditional matching needs to be performed in the historical travel path database according to the specific control strategy. For example, it is necessary to filter travel paths of entering and leaving entertainment venues).

In S250, a corresponding simulated travel path of the affected object is matched for in the historical travel path database based on the corresponding predicted path of the affected object.

The simulated travel path refers to a travel route constrained by the control plan in the future. Optionally, in the historical travel path database, a historical travel path that corresponds to the affected object and is not constrained by the control plan may be used as the simulated travel path in this embodiment. For example, in response to a control plan of leaving the city, a historical travel path of an affected object A in the city is used as a simulated travel path of the affected object A. It is also feasible to determine a target user matching the corresponding predicted path of the affected object in the historical travel path database, and a historical travel path that corresponds to the target user and does not conflict with the control plan is selected as the simulated travel path of the affected object. For example, if a predicted travel path of the affected user A at the current time period has the greatest similarity with a predicted travel path of a target user B at the current time period, then a historical travel path that corresponds to the target user B and is not constrained by the control plan is used as the simulated travel path of the affected user A.

In step S260, a transmission trend of the infectious disease in affected objects and unaffected objects is determined according to the corresponding simulated travel path of the affected object and a corresponding predicted path of the unaffected object.

In this embodiment, the corresponding simulated travel path of the affected object and the corresponding predicted path of the unaffected object are used as input parameters so that the transmission trend of the infectious disease in the affected objects and the unaffected objects is determined.

In this embodiment, in response to multiple control plans, transmission trends under different control plans may be separately predicted to determine the effect of different control plans on the transmission trend, so as to help determine the best control plan.

According to the technical solution of this embodiment of the present disclosure, one or more objects to be predicted are determined, a corresponding predicted path of each object to be predicted is matched for in a historical travel path database, and a transmission trend of the infectious disease in the one or more objects to be predicted is determined based on the predicted travel paths. Since the predicted travel path is obtained through the matching in the historical travel path database, it not needed to perform computation by using various models, thus greatly reducing the computation amount and achieving the technical effect of reducing the computation amount for obtaining the predicted travel path.

### Embodiment Three

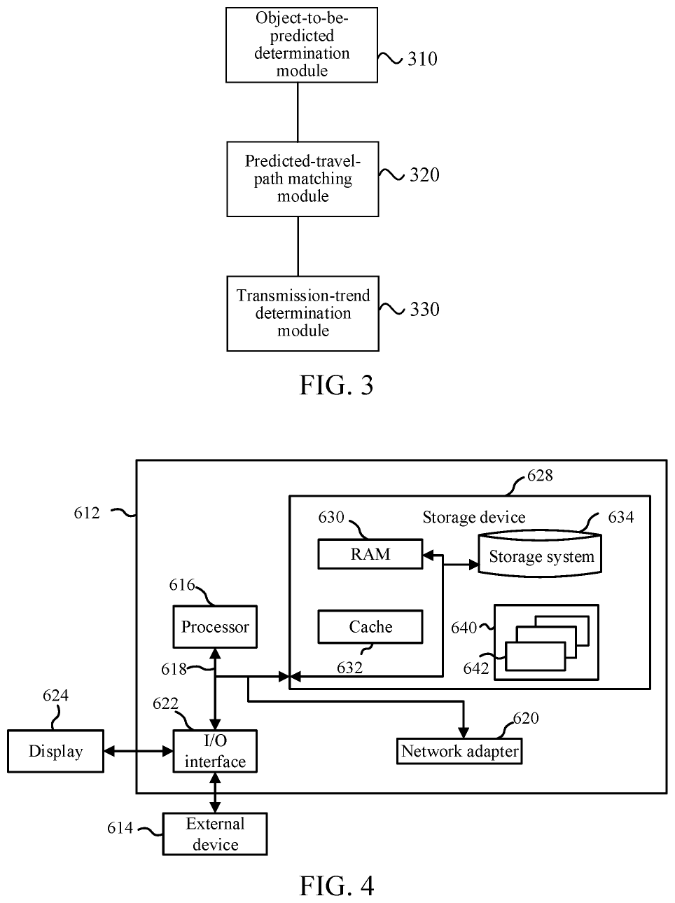

FIG. 3 is a block diagram of an apparatus for predicting transmission of an infectious disease according to Embodiment three of the present disclosure. This embodiment is available to a scenario of predicting a transmission trend of the infectious disease. The apparatus may be implemented in software and/or hardware and may be integrated in a computer apparatus.

As illustrated in FIG. 3, the apparatus for predicting transmission of an infectious disease may include an object-to-be-predicted determination module 310, a predicted-travel-path matching module 320 and a transmission-trend determination module 330.

The object-to-be-predicted determination module 310 is configured to determine one or more objects to be predicted. The predicted-travel-path matching module 320 is configured to match in a historical travel path database for a corresponding predicted path of each object to be predicted. The transmission-trend determination module 330 is configured to determine a transmission trend of the infectious disease in the one or more objects to be predicted based on the predicted travel paths.

Optionally, the historical travel path database includes historical travel paths of different users and corresponding to historical time periods within a time cycle, and the predicted-travel-path matching module 320 includes a time determination unit, a historical-time matching unit and a predicted-travel path matching unit. The time determination unit is configured to obtain a current time period and determine a next time period corresponding to the current time period. The historical-time matching unit is configured to determine a target historical time period matching the next time period in the time cycle. The predicted-travel path matching unit is configured to match in the historical travel path database for a historical travel path corresponding to each object to be predicted in the target historical time period, and use the corresponding historical travel path of each object to be predicted in the target historical time period as the corresponding predicted path of each object to be predicted.

Optionally, the historical travel path database further includes respective identifiers of the different users, and the predicted-travel path matching unit is configured to acquire a target identifier corresponding to each object to be predicted, and determine whether the historical travel path database has a matching identifier matching the target identifier. In response to the historical travel path database having the matching identifier matching the target identifier, the predicted-travel path matching unit determines a historical travel path of a user corresponding to the matching identifier in the target historical time period to serve as the corresponding predicted path of each object to be predicted in the target historical time period. In response to the historical travel path database having no matching identifier matching the target identifier, the predicted-travel path matching unit determines similarity between each of the different users and each object to be predicted, selects a historical travel path corresponding to a user having the greatest similarity in the target historical time period, and determines the historical travel path as the historical travel path corresponding to each object to be predicted in the target historical time period.

Optionally, the apparatus further includes an acquisition module, a historical-travel path determination module and a storage module. The acquisition module is configured to acquire location data of the different users. The historical-travel path determination module is configured to determine location data corresponding to each user to serve as a historical travel path corresponding to each user. The storage module is configured to store the historical travel path corresponding to each user and an identifier corresponding to each user in association according to each historical time period within the time cycle to obtain the historical travel path database.

Optionally, the transmission-trend determination module 330 includes a control-plan acquisition unit, a simulated-travel path determination unit and a transmission-trend determination unit. The control-plan acquisition unit is configured to acquire a pre-made control plan. The simulated-travel path determination unit is configured to determine an affected object and an unaffected object among the one or more objects to be predicted according to the control plan, and match a corresponding simulated travel path of the affected object in the historical travel path database according to a corresponding predicted path of the affected object. The transmission-trend determination unit is configured to determine a transmission trend of the infectious disease in affected objects and unaffected objects according to the corresponding simulated travel path of the affected object and a corresponding predicted path of the unaffected object.

Optionally, the transmission-trend determination module 330 further includes an infected-object determination unit. The infected-object determination unit is configured to determine an infected object, a virus carrier, a recovered patient and an uninfected object among the one or more objects to be predicted, and perform computation on a corresponding predicted path of the infected object, a corresponding predicted path of the virus carrier, a corresponding predicted path of the recovered patient, and a corresponding predicted path of the uninfected object through an SEIR infection model to obtain a transmission trend of the infectious disease in uninfected objects.

Optionally, the apparatus further includes a display module. The display module is configured to display the transmission trend in a visual form.

The apparatus for predicting transmission of an infectious disease provided in this embodiment of the present disclosure can execute the method for predicting transmission of an infectious disease provided in any embodiment of the present disclosure, and has functional modules and beneficial effects corresponding to the execution method. For a complete description of this embodiment, referring to the description of any method embodiment of the present disclosure.

### Embodiment Four

FIG. 4 is a schematic diagram of a computer apparatus according to Embodiment four of the present disclosure. FIG. 4 is a block diagram of an exemplary computer apparatus 612 for implementing an embodiment of the present disclosure. The computer apparatus 612 illustrated in FIG. 4 is merely an example and not intended to limit the function and use scope of this embodiment of the present disclosure.

As illustrated in FIG. 4, the computer apparatus 612 may take a form of a general-purpose computer apparatus. Components of the computer apparatus 612 may include, but are not limited to, one or more processors 616, a storage device 628, and a bus 618 connecting different system components (including the storage device 628 and the one or more processors 616).

The bus 618 represents one or more of several types of bus structures including a storage device bus or a storage device controller, a peripheral bus, a graphics acceleration port, a processor, or a local bus using any one of multiple bus structures. For example, these architectures include, but are not limited to, an industry subversive alliance (ISA) bus, a micro channel architecture (MAC) bus, an enhanced ISA bus, a video electronics standards association (VESA) local bus and a peripheral component interconnect (PCI) bus.

The computer apparatus 612 typically includes multiple computer system readable media. These media may be available media that can be accessed by the computer apparatus 612 and include volatile and non-volatile media, and removable and non-removable media.

The storage device 628 may include a computer system readable medium in the form of a volatile memory, such as a random access memory (RAM) 630 and/or a cache memory 632. The terminal 612 may further include other removable/non-removable and volatile/non-volatile computer system storage media. Just for example, a storage system 634 may be configured to perform reading and writing on a non-removable and non-volatile magnetic medium (not shown in FIG. 4 and usually referred to as a “hard disk driver”). Although not shown in FIG. 4, it is feasible to provide not only a magnetic disk driver for performing reading and writing on a removable non-volatile magnetic disk (for example, a “floppy disk”), but also an optical disk driver for performing reading and writing on a removable non-volatile optical disk, such as a compact disc read-only memory (CD-ROM), a digital video disc-read only memory (DVD-ROM) or other optical media. In these cases, each driver may be connected to the bus 618 via one or more data media interfaces. The storage device 628 may include at least one program product having a group of program modules (for example, at least one program module). These program modules are configured to perform functions of various embodiments of the present disclosure.

A program/utility 642 having a group of program modules 640 (at least one program module 640) may be stored in the storage device 628 or the like. Such program modules 642 include, but are not limited to, an operating system, one or more application programs, other program modules and program data. Each or some combination of these examples may include implementation of a network environment. The program modules 642 generally perform functions and/or methods in embodiments of the present disclosure.

The computer apparatus 612 may communicate with one or more external devices 614 (such as a keyboard, a pointing terminal and a displayer 624). The computer apparatus 612 may communicate with one or more terminals that enable a user to interact with the computer apparatus 612, and/or with any terminal (such as a network card or a modem) that enables the computer apparatus 612 to communicate with one or more other computing terminals. These communications may be performed through an input/output (I/O) interface 622. Moreover, the computer apparatus 612 may communicate with one or more networks (such as a local area networks (LAN), a wide area networks (WAN) and/or a public network, for example, the Internet) through a network adapter 620. As illustrated in FIG. 4, the network adapter 620 communicates with other modules of the computer apparatus 612 via the bus 618. It is to be understood that although not shown in FIG. 5, other hardware and/or software modules may be used in conjunction with the computer apparatus 612. The other hardware and/or software modules include, but are not limited to, microcode, a terminal driver, a redundant processor, an external disk drive array, a redundant arrays of independent disks (RAID) system, a tape driver, a data backup storage system and the like.

The one or more processors 616 execute computer programs stored in storage device 628 to perform various functional applications and data processing, for example, to perform a method for predicting transmission of an infectious disease provided in any embodiment of the present disclosure. The method may include the following operations:

determining one or more objects to be predicted;

matching in a historical travel path database for a corresponding predicted travel path of each of the one or more objects to be predicted; and

determining a transmission trend of the infectious disease in the one or more objects to be predicted based on the predicted travel paths.

According to the technical solution of this embodiment of the present disclosure, one or more objects to be predicted are determined, a corresponding predicted path of each object to be predicted is matched for in a historical travel path database, and a transmission trend of the infectious disease in the one or more objects to be predicted is determined based on the predicted travel paths. Since the predicted travel path is obtained through the matching in the historical travel path database, it not needed to perform computation by using various models, thus greatly reducing the computation amount and achieving the technical effect of reducing the computation amount for obtaining the predicted travel path.

### Embodiment Five

Embodiment five of the present disclosure further provides a computer-readable storage medium storing a computer program. When the computer program is executed by a processor, the method for predicting transmission of an infectious disease provided by any embodiment of the present disclosure is performed. The method may include the following operations:

determining one or more objects to be predicted;

matching in a historical travel path database for a corresponding predicted travel path of each of the one or more objects to be predicted; and

determining a transmission trend of the infectious disease in the one or more objects to be predicted based on the predicted travel paths.

The computer storage medium in this embodiment of the present disclosure may use any combination of one or more computer-readable media. The computer-readable medium may be a computer-readable signal medium or a computer-readable storage medium. The computer-readable storage medium may be, but is not limited to, an electrical, magnetic, optical, electromagnetic, infrared or semiconductor system, apparatus or device, or any combination thereof. More specific examples of the computer-readable storage medium include (non-exhaustive list): an electrical connection having one or more wires, a portable computer magnetic disk, a hard disk, a random access memory (RAM), a read only memory (ROM), an erasable programmable read only memory (EPROM or flash memory), an optical fiber, a portable compact disk read only memory (CD-ROM), an optical memory device, a magnetic memory device, or any suitable combination thereof. In this document, the computer-readable storage medium may be any tangible medium containing or storing a program. The program may be used by or used in conjunction with an instruction execution system, apparatus or device.

The computer-readable signal medium may include a data signal propagated on a base band or as a part of a carrier wave. The data signal carries computer-readable program codes. Such propagated data signals may take multiple forms including, but not limited to, electromagnetic signals, optical signals, or any suitable combination thereof. The computer-readable signal medium may further be any computer-readable medium other than a computer-readable storage medium. The computer-readable medium may send, propagate or transmit the program used by or used in conjunction with the instruction execution system, apparatus or device.

Program codes contained in the computer-readable medium may be transmitted via any suitable medium. The medium includes, but is not limited to, the wireless, a wire, an optical cable, the radio frequency (RF), or any suitable combination thereof.

Computer program codes for performing the operations of the present disclosure may be written in one or more programming languages or a combination thereof. The one or more programming languages include object-oriented programming languages such as Java, Smalltalk and C++, as well as conventional procedural programming languages such as “C” or similar programming languages. The program codes may be executed entirely or partially on a user computer, as a separate software package, partially on the user computer and partially on a remote computer, or entirely on the remote computer or terminal. In the case relate to the remote computer, the remote computer may be connected to the user computer via any kind of network including a local area network (LAN) or a wide area network (WAN), or may be connected to an external computer (for example, via the Internet through an Internet service provider).

According to the technical solution of this embodiment of the present disclosure, one or more objects to be predicted are determined, a corresponding predicted path of each object to be predicted is matched in a historical travel path database, and a transmission trend of the infectious disease in the one or more objects to be predicted is determined based on the predicted travel paths. Since the predicted travel path is obtained through the matching in the historical travel path database, it not needed to perform computation by using various models, thus greatly reducing the computation amount and achieving the technical effect of reducing the computation amount for obtaining the predicted travel path.

It is to be noted that the foregoing merely depicts some illustrative embodiments of the present disclosure and the technical principles used herein. It is to be understood by those skilled in the art that the present disclosure will not be limited to the specific embodiments described herein. Those skilled in the art will be able to make various apparent modifications, adaptations and substitutions without departing from the scope of the present disclosure. Therefore, while the present disclosure has been described in detail in connection with the preceding embodiments, the present disclosure is not limited to the preceding embodiments and may include more other equivalent embodiments without departing from the concept of the present disclosure. The scope of the present disclosure is determined in and by the appended claims.

1. A method for predicting transmission of an infectious disease, the method comprising:
determining one or more objects to be predicted;
matching in a historical travel path database for a corresponding predicted travel path of each of the one or more objects to be predicted; and
determining a transmission trend of the infectious disease in the one or more objects to be predicted based on the predicted travel paths.

2. The method of claim 1, wherein the historical travel path database comprises respective historical travel paths of different users in each historical time period within a time cycle, and wherein matching in a historical travel path database for a corresponding predicted path of each of the one or more objects to be predicted comprises:
obtaining a current time period and determining a next time period corresponding to the current time period;
determining in the time cycle a target historical time period matching the next time period;
matching in the historical travel path database for a corresponding historical travel path of each of the one or more objects to be predicted in the target historical time period; and
using the corresponding historical travel path of each of the one or more objects to be predicted in the target historical time period as the corresponding predicted travel path of each of the one or more objects to be predicted.

3. The method of claim 2, wherein the historical travel path database further comprises respective identifiers of the different users, and wherein matching in a historical travel path database for the corresponding predicted travel path of each of the one or more objects to be predicted comprises:
obtaining a corresponding target identifier of each of the one or more objects to be predicted;
determining whether the historical travel path database comprises a matching identifier that matches the target identifier;
in response to determining that the historical travel path database comprises the matching identifier that matches the target identifier, using the historical travel path of a corresponding user of the matching identifier in the target historical time period as the corresponding predicted travel path of each of the one or more objects to be predicted in the target historical time period; and
in response to determining that the historical travel path database does not comprise a matching identifier that matches the target identifier, determining a similarity between each of the different users and the object to be predicted; and
selecting the corresponding historical travel path of the user having a greatest similarity in the target historical time period, as the corresponding historical travel path of each of the one or more objects to be predicted in the target historical time period.

4. The method of claim 3, further comprising steps as follow prior to matching in the historical travel path database for the corresponding predicted travel path of each of the one or more objects to be predicted:
obtaining respective location data of the different users;
using the corresponding location data of each of the different users as the corresponding historical travel path of each of the different users; and
for each of the historical time periods within the time cycle, storing the corresponding historical travel path of each of the different users and the corresponding identifier of each of the different users in association to obtain the historical travel path database.

5. The method of claim 1, wherein determining a transmission trend of the infectious disease in the one or more objects to be predicted based on the predicted travel paths comprises:
obtaining a pre-made control plan;
determining affected objects and unaffected objects among the one or more objects to be predicted based on the control plan;
matching in the historical travel path database for respective simulated travel paths of the affected objects based on respective predicted travel paths of the affected objects; and
determining the transmission trend of the infectious disease in the affected objects and the unaffected objects based on the respective simulated travel paths of the affected object and the respective predicted travel paths of the unaffected objects.

6. The method of claim 1, wherein determining a transmission trend of the infectious disease in the one or more objects to be predicted based on the predicted travel paths comprises:
determining an infected object, a virus carrier, a recovered patient, and an uninfected object among the one or more objects to be predicted; and
performing computation on a corresponding predicted path of the infected object, a corresponding predicted path of the virus carrier, a corresponding predicted path of the recovered patient, and a corresponding predicted path of the uninfected object using an SEIR (Susceptible, Exposed, Infectious, Recovered) infection model to obtain the transmission trend of the infectious disease in the uninfected objects.

7. The method of claim 1, further comprising displaying the transmission trend in a visual form.

8. The method of claim 5, further comprising displaying the transmission trend in a visual form.

9. The method of claim 6, further comprising displaying the transmission trend in a visual form.

10. An apparatus for predicting transmission of an infectious disease, comprising:
an object-to-be-predicted determination module, configured to determine one or more objects to be predicted;
a predicted-travel-path matching module, configured to match in a historical travel path database for a corresponding predicted path of each of the one or more objects to be predicted; and
a transmission-trend determination module, configured to determine a transmission trend of the infectious disease in the one or more objects to be predicted based on the predicted travel paths.

11. The apparatus of claim 10, wherein the historical travel path database comprises respective historical travel paths of different users in each historical time period within a time cycle, and the predicted-travel-path matching module comprises:
a time determination unit, configured to obtain a current time period and determine a next time period corresponding to the current time period;
a historical-time matching unit, configured to determine in the time cycle a target historical time period matching the next time period; and
a predicted-travel path matching unit, configured to match in the historical travel path database for a corresponding historical travel path of each of the one or more objects to be predicted in the target historical time period, and use the corresponding historical travel path of each of the one or more objects to be predicted in the target historical time period as the corresponding predicted travel path of each of the one or more objects to be predicted.

12. The apparatus of claim 11, wherein the historical travel path database further comprises respective identifiers of the different users, and wherein the predicted-travel path matching unit is configured for:
obtaining a corresponding target identifier of each of the one or more objects to be predicted;
determining whether the historical travel path database comprises a matching identifier that matches the target identifier;
in response to determining that the historical travel path database comprises the matching identifier that matches the target identifier, using the historical travel path of a corresponding user of the matching identifier in the target historical time period as the corresponding predicted travel path of each of the one or more objects to be predicted in the target historical time period; and
in response to determining that the historical travel path database does not comprise a matching identifier that matches the target identifier, determining a similarity between each of the different users and the object to be predicted; and
selecting the corresponding historical travel path of the user having a greatest similarity in the target historical time period, as the corresponding historical travel path of each of the one or more objects to be predicted in the target historical time period.

13. The apparatus of claim 12, further comprising:
an acquisition module, configured to obtain respective location data of the different users;
a historical-travel path determination module, configured to use the corresponding location data of each of the different users as the corresponding historical travel path of each of the different users; and
a storage module, configured to store, for each of the historical time periods within the time cycle, the corresponding historical travel path of each of the different users and the corresponding identifier of each of the different users in association to obtain the historical travel path database.

14. The apparatus of claim 10, wherein the transmission-trend determination module comprises:
a control-plan acquisition unit, configured to obtain a pre-made control plan;
a simulated-travel path determination unit, configured to determine affected objects and unaffected objects among the one or more objects to be predicted based on the control plan, and match in the historical travel path database for respective simulated travel paths of the affected objects based on respective predicted travel paths of the affected objects; and
a transmission-trend determination unit, configured to determine the transmission trend of the infectious disease in the affected objects and the unaffected objects based on the respective simulated travel paths of the affected object and the respective predicted travel paths of the unaffected objects.

15. The apparatus of claim 10, wherein the transmission-trend determination module further comprises an infected-object determination unit configured for:
determining an infected object, a virus carrier, a recovered patient, and an uninfected object among the one or more objects to be predicted; and
performing computation on a corresponding predicted path of the infected object, a corresponding predicted path of the virus carrier, a corresponding predicted path of the recovered patient, and a corresponding predicted path of the uninfected object using an SEIR (Susceptible, Exposed, Infectious, Recovered) infection model to obtain the transmission trend of the infectious disease in the uninfected objects.

16. The apparatus of claim 10, further comprising a display module configured to display the transmission trend in a visual form.

17. A computer apparatus, comprising:
one or more processors; and
a storage device, configured to store one or more computer programs,
which executed by the one or more processors cause the one or more processors to perform the method for predicting transmission of an infectious disease as recited in claim 1.

18. The computer apparatus of claim 17, wherein the historical travel path database comprises respective historical travel paths of different users in each historical time period within a time cycle, and wherein matching in a historical travel path database for a corresponding predicted path of each of the one or more objects to be predicted comprises:
obtaining a current time period and determining a next time period corresponding to the current time period;
determining in the time cycle a target historical time period matching the next time period;
matching in the historical travel path database for a corresponding historical travel path of each of the one or more objects to be predicted in the target historical time period; and
using the corresponding historical travel path of each of the one or more objects to be predicted in the target historical time period as the corresponding predicted travel path of each of the one or more objects to be predicted.

19. A computer-readable storage medium, storing a computer program, which executed by a processor causes the method for predicting transmission of an infectious disease as recited in claim 1.

20. The computer-readable storage medium of claim 19, wherein the historical travel path database comprises respective historical travel paths of different users in each historical time period within a time cycle, and wherein matching in a historical travel path database for a corresponding predicted path of each of the one or more objects to be predicted comprises:
obtaining a current time period and determining a next time period corresponding to the current time period;
determining in the time cycle a target historical time period matching the next time period;
matching in the historical travel path database for a corresponding historical travel path of each of the one or more objects to be predicted in the target historical time period; and
using the corresponding historical travel path of each of the one or more objects to be predicted in the target historical time period as the corresponding predicted travel path of each of the one or more objects to be predicted.
